(19) United States
(12) Patent Application Publication
(10) Pub. No.: US 2025/0195458 A1
(43) Pub. Date: Jun. 19, 2025

(54) SHORT-CHAIN FATTY ACIDS FOR CANCER TREATMENT

(71) Applicant: Ian HODGDON, New Orleans, LA (US)

(72) Inventor: Ian HODGDON, New Orleans, LA (US)

(21) Appl. No.: 18/778,068

(22) Filed: Jul. 19, 2024

Related U.S. Application Data
(63) Continuation of application No. PCT/US2023/011137, filed on Jan. 19, 2023.
(60) Provisional application No. 63/300,923, filed on Jan. 19, 2022.

(51) Int. Cl. A61K 31/19; A61P 35/00

(52) U.S. Cl. CPC A61K 31/19; A61P 35/00

(57) ABSTRACT

This invention is directed to methods for treating and preventing cancer through the administration of a short-chain fatty acid.

This application is a continuation of International Application No. PCT/US2023/011137 filed Jan. 19, 2023, which claims the benefit of U.S. Provisional Application No. 63/300,923, filed Jan. 19, 2022, each of which is entirely incorporated by reference herein.

All patents, patent applications and publications cited herein are hereby incorporated by reference in their entirety. The disclosures of these publications in their entireties are hereby incorporated by reference into this application in order to more fully describe the state of the art as known to those skilled therein as of the date of the invention described and claimed herein.

This patent disclosure contains material that is subject to copyright protection. The copyright owner has no objection to the facsimile reproduction by anyone of the patent document or the patent disclosure as it appears in the U.S. Patent and Trademark Office patent file or records, but otherwise reserves any and all copyright rights.

### FIELD OF THE INVENTION

This invention is directed to compositions and methods for treating or preventing the onset of cancer. The compositions of the invention may comprise short-chain fatty acids.

### BACKGROUND OF THE INVENTION

Cancer is one of the most significant health conditions and has a major impact on society in the United States and across the world. Cancer was the second leading cause of death, after heart disease, in the United States in 2019. In 2019, there were 599,601 cancer deaths; 283,725 were among females and 315,876 among males. The incidence of cancer is widely expected to increase as the US population ages, further augmenting the impact of this condition.

### SUMMARY OF THE INVENTION

Aspects of the invention are drawn to compositions and methods for preventing or treating cancer in a subject. For example, the cancer can be a solid tumor or a liquid cancer. For example, the solid tumor can be colon cancer, pancreatic cancer, or lung cancer. For example, the cancer can be an epithelial based cancer.

Aspects of the invention are also drawn towards compositions and methods for inhibiting the viability of a cancer cell in a subject. For example, the cancer can be a solid tumor or a liquid cancer. For example, the solid tumor can be colon cancer, pancreatic cancer, or lung cancer. For example, the cancer can be an epithelial based cancer.

Embodiments comprise administering to the subject a therapeutically effective amount of one or more short-chain fatty acids. For example, the short-chain fatty acid can comprise 1, 2, 3, 4, 5, or 6 carbon atoms. For example, the short-chain fatty acid can be linear or branched. Non-limiting examples of short-chain fatty acids comprise sodium acetate, sodium propionate, sodium butyrate, formic acid, isobutyric acid, valeric acid, isovaleric acid, 2-methylbutyric acid, or any combination thereof.

Embodiments comprise administering to the subject a combination of short-chain fatty acids, such as two, three, four, five, or more than five short-chain fatty acids. For example, the combination comprises sodium acetate, sodium propionate and sodium butyrate. In such an embodiment, the combination can comprise by weight 60% sodium acetate, 25% sodium propionate and 15% sodium butyrate.

Embodiments comprise administering the short-chain fatty acid orally, parenterally, or topically. For example, parenteral administration comprises intraperitoneal administration or intravenous administration.

Embodiments comprise administering the short-chain fatty acid systemically or locally. For example, locally comprises intratumorally.

Embodiments comprise administering the short-chain fatty acid through a diet supplemented with the short-chain fatty acid.

Embodiments comprise administering a therapeutically effective amount of one or more short-chain fatty acids. For example, the therapeutically effective amount comprises greater than 2000 mg/kg, less than 2000 mg/kg, or about 2000 mg/kg.

In embodiments, the short-chain fatty acid can be administered for one day, five days, one week, one month, or longer than one month.

In embodiments, the short-chain fatty acid can be provided as a pharmaceutical composition comprising a pharmaceutically acceptable carrier, excipient, or diluent.

In embodiments, the short-chain fatty acid can be administered as part of a therapeutic regimen. For example, the therapeutic regimen can comprise chemotherapy, immunotherapy, radiotherapy or surgical therapy.

Aspects of the invention are further drawn to an anti-cancer composition comprising two or more short-chain fatty acids. For example, the anti-cancer compositions comprise two, three, four, five, or more than five short-chain fatty acids. For example, the short-chain fatty acids comprise 1, 2, 3, 4, 5, or 6 carbon atoms, or a combination thereof. For example, the short-chain fatty acids can be linear, branched, or a combination thereof. Non-limiting examples of short-chain fatty acids comprise sodium acetate, sodium propionate, sodium butyrate, formic acid, isobutyric acid, valeric acid, isovaleric acid, 2-methylbutyric acid, or any combination thereof.

In embodiments, the anti-cancer composition can comprise sodium acetate, sodium propionate, sodium butyrate, formic acid, isobutyric acid, valeric acid, isovaleric acid, 2-methylbutyric acid, or any combination thereof. For example, the anti-cancer composition can comprise sodium acetate, sodium propionate and sodium butyrate. For example, the anti-cancer composition comprises by weight 60% sodium acetate, 25% sodium propionate and 15% sodium butyrate.

Other objects and advantages of this invention will become readily apparent from the ensuing description.

### BRIEF DESCRIPTION OF THE FIGURES

FIG. 1 shows graphs depicting the animal weights (g) for both the animal groups treated with short-chain fatty acids and treated with vehicle. Hollow Fibers, loaded in vitro with BxPC-3, Caco-2 or H460 cells, were implanted both subcutaneously and intraperitoneally, into female NMRI nude mice on Day 0. Following randomization on Day 1, animals of Group 2 were treated with 2000 mg/kg SCFA (60% sodium acetate, 25% sodium propionate and 15% sodium butyrate; 3500 mg sodium acetate, 1500 mg sodium propionate, and 1000 mg sodium butyrate), whereas animals of Group 1 were treated with the corresponding Vehicle as described in detail in the corresponding legends. Data are displayed both as means+/−SEM (panel A) and as individual data points for each animal (panel B).

FIG. 2 shows graphs depicting the CellTiter Glo® assay (photons/second) from Hollow Fibers loaded with BxPC-3 cells, implanted both subcutaneously (left) as well as intraperitoneally (right) into female NMRI nude mice on Day 0. Following randomization on Day 1, animals of Group 2 were treated with 2000.0 mg/kg SCFA, whereas animals of Group 1 were treated with the corresponding Vehicle as described in detail herein. Hollow Fibers were collected during necropsy on Day 14 and analyzed using a CellTiter Glo® assay. Data are given both as means+SEM (panel A) and as individual data points together with their corresponding median values and interquartile ranges (panel B). Statistical analysis was performed with unpaired t-test and Mann Whitney test (in parenthesis) and displayed if significant.

FIG. 3 shows graphs depicting the CellTiter Glo® assay (photons/second) from Hollow Fibers loaded with Caco-2 cells, implanted both subcutaneously (left) as well as intraperitoneally (right) into female NMRI nude mice on Day 0. Following randomization on Day 1, animals of Group 2 were treated with 2000.0 mg/kg SCFA, whereas animals of Group 1 were treated with the corresponding Vehicle as described in detail in the corresponding legends. Hollow Fibers were collected during necropsy on Day 14 and analyzed using a CellTiter Glo® assay. Data are given both as means+SEM (panel A) and as individual data points together with their corresponding median values and interquartile ranges (panel B). Statistical analysis was performed with unpaired t-test and Mann Whitney test (in parenthesis) and displayed if significant.

FIG. 4 shows graphs depicting the CellTiter Glo® assay (photons/second) from Hollow Fibers loaded with H460 cells, implanted both subcutaneously (left) as well as intraperitoneally (right) into female NMRI nude mice on Day 0. Following randomization on Day 1, animals of Group 2 were treated with 2000.0 mg/kg SCFA, whereas animals of Group 1 were treated with the corresponding Vehicle as described in detail herein. Hollow Fibers were collected during necropsy on Day 14 and analyzed using a CellTiter Glo® assay. Data are given both as means+SEM (panel A) and as individual data points together with their corresponding median values and interquartile ranges (panel B). Statistical analysis was performed with unpaired t-test and Mann Whitney test (in parenthesis) and displayed if significant.

FIG. 5 shows a graph depicting the CellTiter Glo® assay from Hollow Fibers loaded with H460 cells (lung), Caco-2 cells (colon), and BxPC-3 (pancreatic) cells, implanted both subcutaneously (SQ) or intraperitoneally (IP). Following randomization on Day 1, animals were treated with 2000.0 mg/kg SCFA or vehicle control, with a decrease in cancer activity in the treatment group compared to the control group illustrated in the graph.

FIG. 6 shows a graph depicting the weight (g) of animals treated with vehicle control.

FIG. 7 shows a graph depicting the weight (g) of animals treated with 2000.0 mg/kg SCFA.

FIG. 8 shows a graph comparing the mean animal weight of animals treated with vehicle control to that of animals treated with 2000.0 mg/kg SCFA.

### DETAILED DESCRIPTION OF THE INVENTION

The invention is based upon the discovery that short-chain fatty acids are effective as a cancer therapeutic approach. The results presented herein demonstrate that the administration of short-chain fatty acids to subjects having cancer is effective in preventing and treating cancer.

Detailed descriptions of one or more embodiments are provided herein. It is to be understood, however, that the invention can be embodied in various forms. Therefore, specific details disclosed herein are not to be interpreted as limiting, but rather as a basis for the claims and as a representative basis for teaching one skilled in the art to employ the present invention in any appropriate manner.

The singular forms “a”, “an” and “the” include plural reference unless the context dictates otherwise. The use of the word “a” or “an” when used in conjunction with the term “comprising” in the claims and/or the specification may refer to “one,” but it is also consistent with the meaning of “one or more,” “at least one,” and “one or more than one.”

Wherever any of the phrases “for example,” “such as,” “including” and the like are used herein, the phrase “and without limitation” is understood to follow unless explicitly stated otherwise. Similarly, “an example,” “exemplary” and the like are understood to be nonlimiting.

The term “substantially” allows for deviations from the descriptor that do not negatively impact the intended purpose. Descriptive terms are understood to be modified by the term “substantially” even if the word “substantially” is not explicitly recited.

The terms “comprising” and “including” and “having” and “involving” (and similarly “comprises”, “includes,” “has,” and “involves”) and the like are used interchangeably and have the same meaning. Specifically, each of the terms is defined consistent with the common United States patent law definition of “comprising” and is therefore interpreted to be an open term meaning “at least the following,” and is also interpreted not to exclude additional features, limitations, aspects, etc. Thus, for example, “a process involving steps a, b, and c” can refer to the process including at least steps a, b and c. Wherever the terms “a” or “an” are used, “one or more” is understood, unless such interpretation is nonsensical in context.

As used herein, the term “about” can refer to approximately, roughly, around, or in the region of. When the term “about” is used in conjunction with a numerical range, it modifies that range by extending the boundaries above and below the numerical values set forth. In general, the term “about” is used herein to modify a numerical value above and below the stated value by a variance of 20 percent up or down (higher or lower).

Throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6, etc., as well as individual numbers within that range, for example, 1, 2, 2.7, 3, 4, 5, 5.3, 6, and any whole and partial increments therebetween. This applies regardless of the breadth of the range.

Aspects of the invention are directed towards the administration of short-chain fatty acids to a subject for the prevention and/or treatment of cancer.

A “short-chain fatty acid” can refer to fatty acids (also referred to as fatty acid compounds) comprising 1 to 6 carbon atoms. For example, the short-chain fatty acid can have a chain length ranging from 1 to 6 carbon atoms. In embodiments, the short-chain fatty acid has one carbon atom. In another embodiment, the short-chain fatty acid has two carbon atoms. In another embodiment, the short-chain fatty acid has three carbon atoms. In yet another embodiment, the short-chain fatty acid has four carbon atoms. In another embodiment, the short-chain fatty acid has five carbon atoms. In yet another embodiment, the short-chain fatty acid has six carbon atoms. In embodiments, the short-chain fatty acid can be linear or branched.

The skilled artisan will recognize that any fatty acid having 1-6 carbon atoms can be utilized in compositions and methods as described herein. Non-limiting examples of such short-chain fatty acids comprise acetic acid, propionic acid, butyric acid, isobutyric acid, caproic acid, sodium acetate, sodium propionate, sodium butyrate, formic acid, isobutyric acid, valeric acid, isovaleric acid, and 2-methylbutyric acid, and analogs and derivatives thereof. For example, non-limiting examples of short-chain fatty acids comprise:

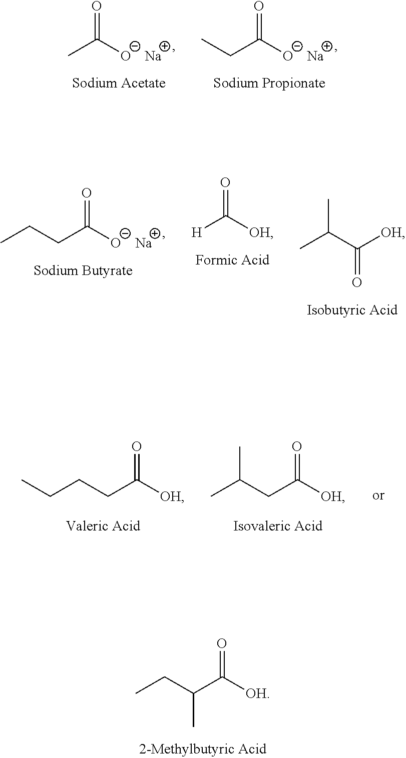

Embodiments can comprise a derivative or analog of a short chain fatty acid. A “derivative” can refer to chemical substance derived from another substance either directly or by modification or partial substitution, and can include differences in one atom, element or group or more than one atom, element or group. For example, compounds can be derivatized as the corresponding salts, esters, enol ethers, enol esters, acetals, ketals, orthoesters, hemiacetals, hemiketals, acids, bases, solvates, hydrates or prodrugs thereof, prior to formulation, as described above. An “analog” can refer to a chemical compound that has a similar structure and similar chemical properties to those of another compound, but differs from it by a single atom, element or group. Such derivatives and/or analogs can be readily prepared by those of skill in this art using known methods for such derivatization.

Embodiments can be provided as a salt. For example, the short-chain fatty acid or analog or derivative thereof can be provided as a salt. “Salts” or “pharmaceutically acceptable salts” can refer to a salt prepared by combining a compound of the invention, such as a short chain fatty acid, with an acid whose anion, or a base whose cation, can be suitable for human consumption. Non-limiting examples of pharmaceutically acceptable salts comprise mineral acid salts, such as hydrochlorides, hydrobromides, phosphates and sulphates, or salts of organic acids, such as acetates, propionates, malonates and benzoates. Pharmaceutically acceptable salts can include, but are not limited to, amine salts, such as but not limited to N,N′-dibenzylethylenediamine, chloroprocaine, choline, ammonia, diethanolamine and other hydroxyalkylamines, ethylenediamine, N-methylglucamine, procaine, N-benzylphenethylamine, 1-para-chlorobenzyl-2-pyrrolidin-1′-ylmethylbenzimidazole, diethylamineand other alkylamines, piperazine and tris(hydroxymethyl)aminomethane; alkali metal salts, such as but not limited to lithium, potassium and sodium; alkali earth metal salts, such as but not limited to barium, calcium and magnesium; transition metal salts, such as but not limited to zinc; and other metal salts, such as but not limited to sodium hydrogen phosphate and disodium phosphate; and also including, but not limited to, salts of mineral acids, such as but not limited to hydrochlorides and sulfates; and salts of organic acids, such as but not limited to acetates, lactates, malates, tartrates, citrates, ascorbates, succinates, butyrates, valerates and fumarates

Aspects of the invention can also be drawn to pharmaceutical compositions comprising one or more short-chain fatty acids for the prevention and/or treatment of cancer. The terms “pharmaceutical composition” or a “pharmaceutical formulation” can refer to a composition comprising one or more short-chain fatty acids and a pharmaceutically acceptable carrier, excipient or diluent. Accordingly, such compositions can be suitable for diagnostic, therapeutic, or preventive use in vitro, in vivo, or ex vivo.

Embodiments can be sterile and can be free of contaminants that can elicit an undesirable response within the subject. For example, the pharmaceutical composition can be sterile and can be free of contaminants that can elicit an undesirable response within the subject. For example, the short-chain fatty acid and/or other active agents in the pharmaceutical composition can be pharmaceutical grade. Embodiments, such as the pharmaceutical compositions, can be designed for administration to subjects or patients in need thereof via a number of different routes of administration including those described herein, such as oral, intravenous, buccal, rectal, parenteral, intraperitoneal, intradermal, intratracheal, intramuscular, subcutaneous, inhalational and the like.

Embodiments, such as the short-chain fatty acid or pharmaceutical composition, can be administered alone, but can also be administered with other compounds, excipients, fillers, binders, carriers, or other vehicles selected based upon the chosen route of administration and standard pharmaceutical practice. Administration can be by way of carriers or vehicles, such as injectable solutions, including sterile aqueous or non-aqueous solutions, or saline solutions; creams; lotions; capsules; tablets; granules; pellets; powders; suspensions, emulsions, or microemulsions; patches; micelles; liposomes; vesicles; implants, including microimplants; eye drops; other proteins and peptides; synthetic polymers; microspheres; nanoparticles; and the like.

Embodiments, such as pharmaceutical compositions comprising one or more short chain fatty acids, can comprise a pharmaceutically acceptable excipient, carrier, or diluent. “Pharmaceutically acceptable” can refer to those properties and/or substances which are acceptable to the patient from a pharmacological/toxicological point of view and to the manufacturing pharmaceutical chemist from a physical/chemical point of view regarding composition, formulation, stability, patient acceptance and bioavailability.

A “pharmaceutically acceptable excipient,” “pharmaceutically acceptable diluent,” “pharmaceutically acceptable carrier,” or “pharmaceutically acceptable adjuvant” can be useful in preparing a pharmaceutical composition that is safe, non-toxic and neither biologically nor otherwise undesirable, and can include an excipient, diluent, carrier, and adjuvant that is acceptable for veterinary use and/or human pharmaceutical use. A “pharmaceutically acceptable excipient, diluent, carrier and/or adjuvant” can include one and more such excipients, diluents, carriers, and adjuvants.

The term “pharmaceutically acceptable carrier” can refer to a pharmaceutically acceptable material, composition, or carrier, such as a liquid or solid filler, stabilizer, dispersing agent, suspending agent, diluent, excipient, thickening agent, solvent or encapsulating material, involved in carrying or transporting a compound useful within the invention within or to the patient such that it can perform its intended function. For example, such constructs are carried or transported from one organ, or portion of the body, to another organ, or portion of the body. Each carrier can be “acceptable” in the sense of being compatible with the other ingredients of the formulation, including the compound useful within the invention, and not injurious to the patient. Some examples of materials that can serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; surface active agents; alginic acid; pyrogen-free water; isotonic saline; Ringer's solution; ethyl alcohol; phosphate buffer solutions; and other non-toxic compatible substances employed in pharmaceutical formulations.

A “pharmaceutically acceptable carrier” can also include any and all coatings, antibacterial and antifungal agents, and absorption delaying agents, and the like that are compatible with the activity of the compound useful within the invention and are physiologically acceptable to the patient. Supplementary active compounds can also be incorporated into the compositions. The “pharmaceutically acceptable carrier” can further include a pharmaceutically acceptable salt of the compound useful within the invention. Other additional ingredients that can be included in the pharmaceutical compositions used in the practice of the invention are known in the art and described, for example in Remington's Pharmaceutical Sciences (Genaro, Ed., Mack Publishing Co., 1985, Easton, PA), which is incorporated herein by reference.

Non-limiting examples of pharmaceutically acceptable carriers, excipients, binders and fillers include glucose, lactose, gum acacia, gelatin, mannitol, xanthan gum, locust bean gum, galactose, oligosaccharides and/or polysaccharides, starch paste, magnesium trisilicate, talc, corn starch, starch fragments, keratin, colloidal silica, potato starch, urea, dextrans, dextrins, and the like. For example, the pharmaceutically acceptable carriers, excipients, binders, and fillers that can be used include those which render the short chain fatty acids amenable to intranasal delivery, oral delivery, parenteral delivery, intravitreal delivery, intraocular delivery, ocular delivery, subretinal delivery, intrathecal delivery, intravenous delivery, subcutaneous delivery, transcutaneous delivery, intracutaneous delivery, intracranial delivery, topical delivery, and the like. Moreover, the packaging material can be biologically inert or lack bioactivity, such as plastic polymers, and silicone, and can be processed internally by the subject without affecting the effectiveness of the composition/formulation packaged and/or delivered therewith.

Embodiments, such as the short-chain fatty acids and/or pharmaceutical composition, can be included, or packaged, with non-toxic compounds, such as pharmaceutically acceptable carriers, excipients, diluents, binders and fillers.

Embodiments, such as the short-chain fatty acid and/or pharmaceutical composition, can be formulated as oily suspensions in a pharmaceutically acceptable carrier such as a soft gelatin capsule whereby the active ingredient(s) is (are) mixed with an aqueous solution or oil medium, including suspending the active ingredient in a plant seed derived or vegetable oil, for example hemp seed oil, evening primrose seed oil, borage seed oil, olive oil, sesame oil or coconut oil. The oily suspensions can contain a thickening agent or other agent necessary to produce a commercially viable product, such as, beeswax, paraffin, lecithin, or cetyl alcohol. Sweetening and flavoring agents can be added to provide a palatable oral composition. These compositions can be preserved by the addition of an antioxidant such as ascorbic acid.

Embodiments, such as the short-chain fatty acid and/or pharmaceutical composition, can be sterilized and/or mixed with auxiliary agents and other excipients including but not limited to other carrier oils, preservatives, glycerins, stabilizers, waxes, wetting agents, emulsifiers, suspending agents, lecithin, esters or partial esters, buffers, coloring agents, flavorings and/or aromatic substances and the like which do not deleteriously react with the active compounds.

Embodiments, such as the short-chain fatty acid and/or pharmaceutical composition, can be formulated as a dietary supplement for subject with cancer or at risk of cancer. “Dietary supplement” can refer to a small amount of a short-chain fatty acid or pharmaceutical composition comprising the same for supplementation of a subject's diet. In embodiments, the dietary supplement can be packaged in single or multiple does units. Dietary supplements do not generally provide significant amounts of calories but may contain other micronutrients (e.g., vitamins or minerals). However, in embodiments, dietary supplements can be provided in combination with a source of calories. For example, such dietary supplements can be meal replacements or supplements (e.g., nutrient or energy bars or nutrient beverages or concentrates).

Embodiments, such as the short-chain fatty acid and/or pharmaceutical composition, can be formulated into preparations for injection by dissolving, suspending, or emulsifying them in an aqueous or non-aqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers, and preservatives.

Embodiments, such as the short-chain fatty acid and/or pharmaceutical composition, can be utilized in aerosol formulation to be administered via inhalation. For example, one or more short-chain fatty acids or pharmaceutical composition can be formulated into pressurized acceptable propellants such as dichlorodifluoromethane, propane, nitrogen, and the like.

Embodiments, such as the short-chain fatty acid and/or pharmaceutical composition, can be formulated for delivery by a continuous delivery system. The term “continuous delivery system” can be used interchangeably herein with “controlled delivery system” and encompasses continuous (e.g., controlled) delivery devices (e.g., pumps) in combination with catheters, injection devices, and the like, a wide variety of which are known in the art.

Embodiments can also provide packaged short-chain fatty acids or pharmaceutical composition(s) for prevention, restoration, or use in treating the disease or condition, such as cancer. The packaged embodiments can further comprise one or more of: instructions for using the short-chain fatty acid or pharmaceutical composition to treat or prevent the cancer. The kit can further include buffers and reagents known in the art for administering various combinations of the components described herein to the subject.

Embodiments, such as the therapeutically effective amount of one or more short chain fatty acids or pharmaceutical compositions, can be calibrated in order to adapt both to different individuals and to the different needs of a single individual. For example, embodiments need not counter every cause in every individual. Rather, by countering the necessary causes, embodiments described herein can restore the body and brain to their normal function. Then the body and brain themselves can correct the remaining deficiencies. No drug can correct every single aspect of cancer, but the embodiments described herein can maximize the possibility.

Embodiments, such as the short-chain fatty acid or pharmaceutical compositions, can be administered to the subject using any means capable of resulting in the desired effect, for example, preventing or treating cancer. Thus, embodiments can be incorporated into a variety of formulations for therapeutic administration. For example, the short-chain fatty acid or pharmaceutical composition can be formulated into pharmaceutical compositions by combination with appropriate, pharmaceutically acceptable carriers or diluents, and can be formulated into preparations in solid, semi-solid, liquid or gaseous forms, such as tablets, capsules, powders, granules, ointments, solutions, suppositories, injections, inhalants, and aerosols. In any event, embodiments can contain a quantity of at least one short-chain fatty acid or pharmaceutical composition adequate to achieve the desired state in the subject being treated. Methods of preparing such dosage forms are known, or will be apparent upon consideration of this disclosure, to those skilled in the art. See, e.g., Remington's Pharmaceutical Sciences, Mack Publishing Company, Easton, Pennsylvania, 17th edition, 1985.

Embodiments, such as the short-chain fatty acid and/or pharmaceutical composition, can include those that comprise a sustained release matrix. A sustained-release matrix can refer to a matrix made of materials, for example polymers, which are degradable by enzymatic or acid-based hydrolysis or by dissolution. Once inserted into the body, the matrix can be acted upon by enzymes and body fluids. A sustained-release matrix can be chosen from biocompatible materials such as liposomes, polylactides (polylactic acid), polyglycolide (polymer of glycolic acid), polylactide co-glycolide (copolymers of lactic acid and glycolic acid), polyanhydrides, poly(ortho) esters, polypeptides, hyaluronic acid, collagen, chondroitin sulfate, carboxylic acids, fatty acids, phospholipids, polysaccharides, nucleic acids, polyamino acids, amino acids such as phenylalanine, tyrosine, isoleucine, polynucleotides, polyvinyl propylene, polyvinylpyrrolidone and silicone. Non-limiting examples of biodegradable matrices can include a polylactide matrix, a polyglycolide matrix, and a polylactide co-glycolide (co-polymers of lactic acid and glycolic acid) matrix. In addition, embodiments can be used in conjunction with other treatments that use sustained-release formulations.

Embodiments, such as the short-chain fatty acid and/or pharmaceutical composition, can include those that can be delivered in a controlled release system. For example, one or more short chain fatty acids or pharmaceutical compositions comprising the same can be administered using intravenous infusion, an implantable osmotic pump, a transdermal patch, liposomes, or other modes of administration. In one embodiment, a pump may be used (Sefton (1987). CRC Crit. Ref. Biomed. Eng. 14:201; Buchwald et al. (1980). Surgery 88:507; Saudek et al. (1989). N. Engl. J. Med. 321:574). In another embodiment, polymeric materials are used. In embodiments, a controlled release system is placed in proximity of the therapeutic target, for example a cancer cell or a solid tumor, thus requiring only a fraction of the systemic dose. Other controlled release systems are discussed in the review by Langer (1990). Science 249:1527-1533.

Embodiments, such as the short-chain fatty acid and/or pharmaceutical composition, can be part of a delayed-release formulation. For example, one or more short chain fatty acids or pharmaceutical compositions comprising the same can be a part of a delayed-release formulation. Delayed-release dosage formulations can be prepared as described in standard references such as “Pharmaceutical dosage form tablets”, eds. Liberman et. al. (New York, Marcel Dekker, Inc., 1989), “Remington—The science and practice of pharmacy”, 20th ed., Lippincott Williams & Wilkins, Baltimore, MD, 2000, and “Pharmaceutical dosage forms and drug delivery systems”, 6th Edition, Ansel et al., (Media, PA: Williams and Wilkins, 1995). These references provide information on excipients, materials, equipment, and process for preparing tablets and capsules and delayed release dosage forms of tablets, capsules, and granules. These references provide information on carriers, materials, equipment, and process for preparing tablets and capsules and delayed release dosage forms of tablets, capsules, and granules.

Aspects of the invention are also drawn towards therapeutic combinations of two or more short-chain fatty acids. The term “in combination” does not restrict the order in which the two or more short-chain fatty acids can be administered to a subject, or the route of administration. For example, the two or more short-chain fatty acids can be administered to the subject as a single combination composition or can be administered as separate compositions. If administered separately, the two or more short-chain fatty acids or pharmaceutical compositions comprising the same can be administered simultaneously, or sequentially, as described herein.

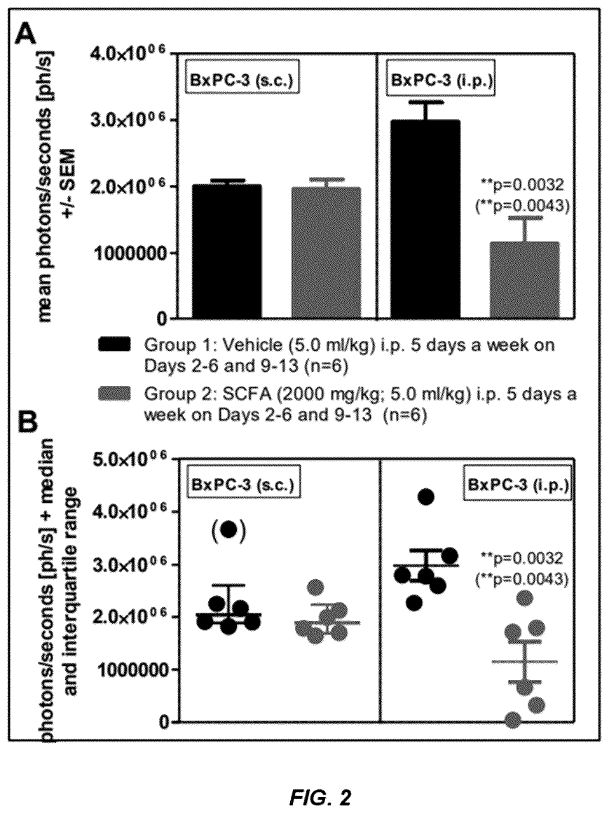

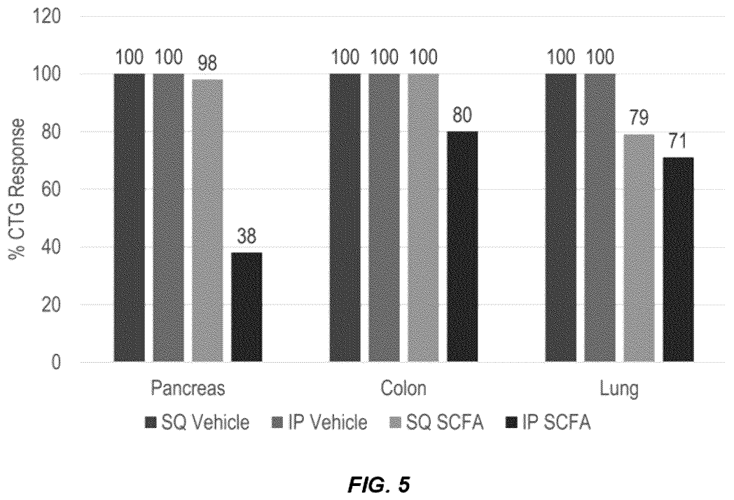

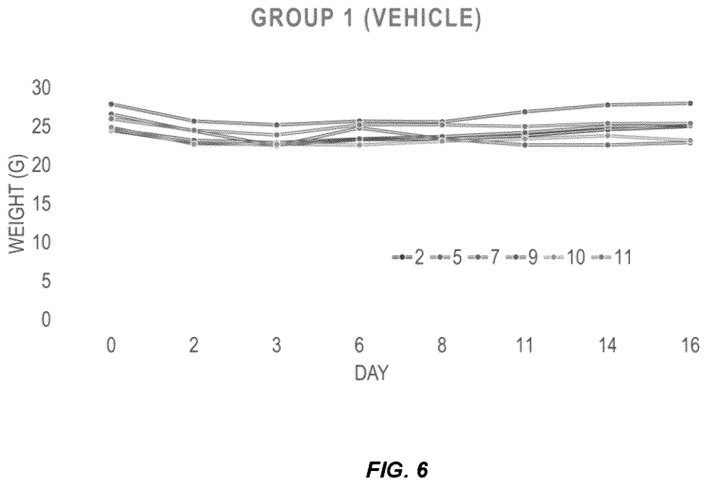

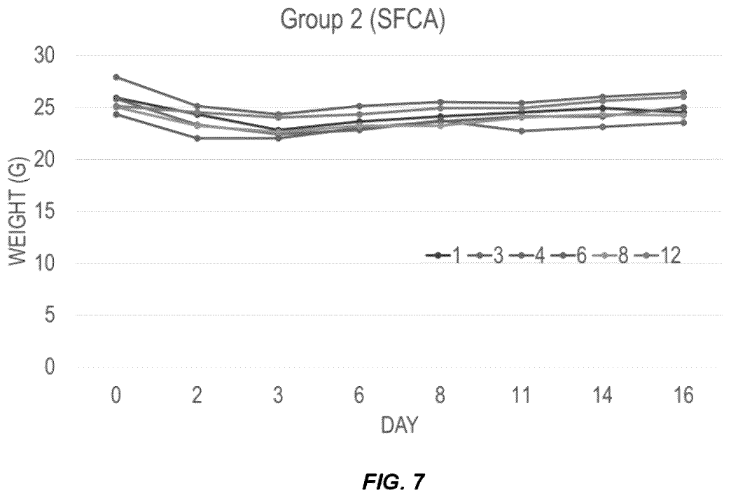

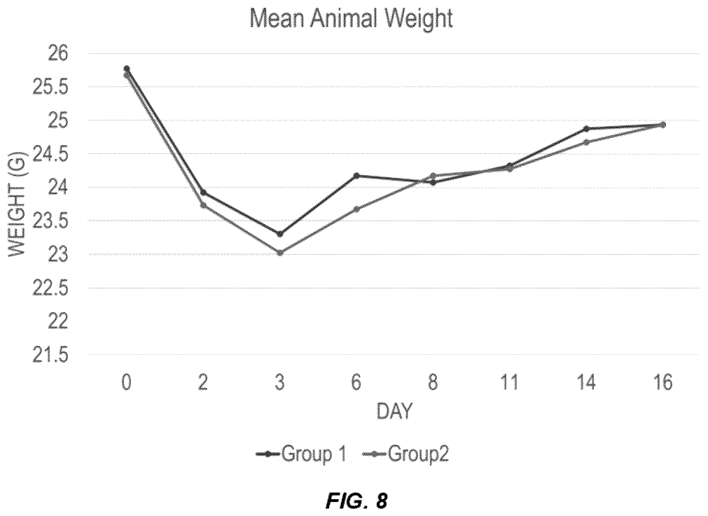

A “combination composition” (which can also be referred to as a “combination pharmaceutical composition”) can refer to a pharmaceutical composition which comprises a mixture of at least two different short-chain fatty acids. For example, the mixture can comprise at least two different fatty acids with 1-6 carbon atoms. For example, the mixture can comprise two or more of acetic acid, propionic acid, butyric acid, isobutyric acid, caproic acid, sodium acetate, sodium propionate, sodium butyrate, formic acid, isobutyric acid, valeric acid, isovaleric acid, and 2-methylbutyric acid, and analogs and derivatives thereof. Referring to FIGS. 2-8, for example, embodiments can comprise a combination composition comprising sodium acetate, sodium propionate, and sodium butyrate.

Embodiments can comprise any mixture of two or more short-chain fatty acids (e.g., fatty acids with 1-6 carbon atoms), wherein amounts of the two or more short-chain fatty acids adds up to 100 wt % of the composition. For example, the combination composition can comprise any amount of a first short-chain fatty acid, any amount of a second short-chain fatty acid, any amount of a third short-chain fatty acid, and so on, such that the total adds up to 100 wt % of the total of the composition. Referring to FIGS. 2-8, for example, the combination composition can comprise 60% sodium acetate, 25% sodium propionate and 15% sodium butyrate. In embodiments, the mixture of two or more short-chain fatty acids can be provided as a ratio. Referring to FIGS. 2-8, for example, the combination composition can comprise a ratio of 6:2.5:1.5 of sodium acetate, sodium propionate, and sodium butyrate, respectively.

Embodiments of the combination composition can be prepared by mixing any combination of two or more short-chain fatty acids (e.g., a fatty acid with 1-6 carbon atoms) together with one or more pharmaceutically acceptable excipients, carriers, or diluents. Such combination compositions can be used for prevention, treatment, control or slow down of cancer and/or its symptoms. For example, the combination composition can comprise two or more fatty acids with 1-6 carbon atoms. For example, the combination composition can comprise three or more fatty acids with 1-6 carbon atoms. For example, the combination composition can comprise four or more fatty acids with 1-6 carbon atoms. For example, the combination composition can comprise five or more fatty acids with 1-6 carbon atoms. For example, the combination composition can comprise more than five fatty acids with 1-6 carbon atoms.

Without wishing to be bound by theory, combination compositions can produce a synergistic effect/action. “Synergy” can refer to an effect produced by a combination (e.g., of two or more short-chain fatty acids) that is greater than the expected additive effectives of the combination components. In embodiments, “synergy” between two or more short-chain fatty acids can result in increased anti-cancer activity. As used herein, “synergy” or “synergistic interactions” can refer to the interaction or cooperation of two or more short-chain fatty acids to produce a combined effect greater than the sum of their separate effects.

Aspects of the invention are directed towards methods of preventing, treating, ameliorating a symptom of, or managing cancer in a subject. In embodiments, the method comprises administering to the subject a therapeutically effective amount of a short-chain fatty acid or a pharmaceutical composition comprising the same.

The terms “patient,” “subject,” “individual,” and the like can be used interchangeably herein, and can refer to any animal, or cells thereof whether in vitro or in situ, amenable to the methods described herein. In embodiments, the patient, subject or individual is a human. “Subject,” “individual,” or “patient,” can refer to a vertebrate, such as a mammal. Mammals can include, but are not limited to, murines, simians, humans, farm animals, sport animals, and pets. The term “pet” can include a dog, cat, guinea pig, mouse, rat, rabbit, ferret, and the like. The term “farm animal” can include a horse, sheep, goat, chicken, pig, cow, donkey, llama, alpaca, turkey, and the like.

The terms “treatment” and “treating” can refer to the management and care of a subject for the purpose of combating cancer, in any manner in which one or more of the symptoms of cancer are ameliorated or otherwise beneficially altered. The term can include the full spectrum of treatments the cancer from which the patient is suffering, such as administration of the active compound for the purpose of: alleviating or relieving symptoms or complications; delaying the progression of cancer; curing or eliminating the cancer; and/or preventing the cancer.

The terms “preventing” or “prevention” can refer to the management and care of a subject for the purpose of hindering the development of the cancer and includes the administration of one or more short-chain fatty acids or pharmaceutical compositions comprising the same to prevent or reduce the risk of the onset of symptoms or complications.

The term “treatment of cancer” or “treating cancer” can refer to the prevention or alleviation or amelioration of any of the phenomena known in the art to be associated with the pathology commonly known as “cancer.” The term “cancer” can refer to the spectrum of pathological symptoms associated with the initiation or progression, as well as metastasis, of malignant tumors. The term “tumor” can refer to a new growth of tissue in which the multiplication of cells is uncontrolled and progressive. In embodiments, the tumor can be a malignant tumor, one in which the primary tumor has the properties of invasion or metastasis or which shows a greater degree of anaplasia than do benign tumors. Thus, “treatment of cancer” or “treating cancer” can refer to an activity that prevents, alleviates or ameliorates any of the primary phenomena (initiation, progression, metastasis) or secondary symptoms associated with the disease.

Treating cancer can be indicated by, for example, inhibiting or delaying invasiveness of a cancer. “Cancer invasion” can refer to the movement caused by cancer cells in vivo, into or through biological tissue or the like. For example, movements caused by cancer cells into or through barriers formed by special cell-based proteins, such as collagen and Matrigel, and other substances.

“Preventing cancer” can refer to prevention of cancer occurrence. In embodiments, the preventative treatment reduces the recurrence of the cancer. In other embodiments, preventative treatment decreases the risk of a patient from developing a cancer, or inhibits progression of a pre-cancerous state (e.g., a colon polyp) to actual malignancy.

Aspects of the invention are also drawn towards methods for inhibiting the viability of a cancer cell. Referring to FIGS. 2, 3 and 4, for example, treatment with short chain fatty acid can reduce cell viability of pancreatic, lung, and colorectal cancers, respectively. The term “cell viability” can refer to a determination of living (viable) or dead cells based on a total cell sample in any given population. For example, the population can be a population of cells cultured in vitro. A cell is considered viable if it has the ability to grow and develop. Viability assays are based on either the physical properties of viable cells such as membrane integrity or on their metabolic activity. Cell viability can also be estimated by measuring the rate of cell proliferation of the entire population which represents the overall balance of the rates of cell growth and cell death. Rates of cell growth can be directly measured, by counting the number of cells, and by using any number of commercially available cell proliferation assays which directly scores the rate of cell growth.

“Reduced cell viability” can refer to a level of cell viability that is reduced in a first cell population compared to a second cell population. In embodiments, the level of cell viability in the first cell population can be reduced by at least 5% compared to the level of cell viability in the second cell population. In embodiments, the level of cell viability in the first cell population can be reduced by at least 5%, 6%, 7%, 8%, 9%, 10%, 15%, 20%, 25%, 30%, 35%, 40%, 50%, 60%, 70%, 80%, 90% or more compared to the level of cell viability in the second cell population. In embodiments, cell viability in the first cell population is completely inhibited compared to cell viability in the second cell population.

Aspects of the invention are also drawn to managing a subject afflicted with or at risk of cancer. The terms “manage,” “managing,” and “management,” in the context of the administration of a therapy to a subject, can refer to the beneficial effects that a subject derives from a therapy, such as a short-chain fatty acid or pharmaceutical composition comprising the same, which does not result in a cure of the cancer. In embodiments, a subject is administered one or more therapies to manage the cancer so as to prevent the progression or worsening of the cancer.

“Cancer” can refer to diseases in which abnormal cells divide without control and can invade nearby tissues. Cancer cells can also spread to other parts of the body through the blood and lymph systems. Unregulated cell division and growth results in the formation of malignant tumors that invade neighboring tissues and may also metastasize to distant parts of the body through the lymphatic system or bloodstream. A “cancer” or “cancer tissue” can include a tumor. In embodiments, the disclosure provides a pharmaceutical composition for the treatment of cancer. There are several main types of cancer. Carcinoma is a cancer that begins in the skin or in tissues that line or cover internal organs. Sarcoma is a cancer that begins in bone, cartilage, fat, muscle, blood vessels, or other connective or supportive tissue. Leukemia is a cancer that starts in blood-forming tissue, such as the bone marrow, and causes large numbers of abnormal blood cells to be produced and enter the blood. Lymphoma and multiple myeloma are cancers that begin in the cells of the immune system. Central nervous system cancers are cancers that begin in the tissues of the brain and spinal cord. Also called malignancy.

In embodiments, the cancer can comprise a solid tumor or a liquid cancer. A “solid tumor” can refer to an abnormal mass of tissue that does not contain cysts or liquid. A “non-solid tumor”, which can be referred to as a “liquid cancer”, can refer to neoplasia of the hemopoietic system, such as lymphoma, myeloma, and leukemia, or neoplasia without solid formation generally and with spread substantially.

In embodiments, the solid tumors can include but is not limited to brain cancer, lung cancer, liver cancer, hepatocellular carcinoma (HCC), esophageal cancer, cholangiocarcinoma, gallbladder carcinoma, stomach cancer, abdominal cancer, small bowel cancer, gastrointestinal cancer, gastric cancer, pancreatic cancer, renal cell carcinoma, renal cancer, bone cancer, ocular cancer, oral cancer, bile duct cancer, gallbladder cancer, testicular cancer, spleen cancer, adrenal cancer, breast cancer, ovarian cancer, uterine cancer, cervical cancer, endometrial cancer, colorectal cancer, colon cancer, rectal cancer, bladder cancer, superficial bladder cancer, prostate cancer, adrenal tumors, squamous cell carcinoma, neuroma, malignant neuroma, myoepithelial carcinoma, synovial sarcoma, rhabdomyosarcoma, gastrointestinal interstitial cell tumor, skin cancer, basal cell carcinoma, malignant melanoma, thyroid cancer, nasopharyngeal carcinoma, hemangioma, epidermoid carcinoma, head and neck cancer, glioma, or Kaposi's sarcoma. In embodiments, the solid tumor comprises colon cancer, pancreatic cancer, or lung cancer.

In embodiments, the non-solid tumors can include but are not limited to leukemia, acute leukemia, chronic leukemia, chronic myelocytic leukemia, acute myelogenous leukemia, chronic myelogenous leukemia, chronic lymphocytic leukemia, acute lymphoblastic leukemia, T-cell leukemia, hairy cell leukemia, polycythemia, myelodysplastic syndrome, multiple myeloma, lymphadenoma, Hodgkin's lymphoma, and Non-Hodgkin's lymphoma.

In embodiments, the cancer can comprise an epithelial-based cancer. The terms “epithelial-based cancer” or “epithelial-based tumor” can refer to any cancer that begins in the cells of the internal or external lining of the body. In embodiments, “epithelial-based cancer” can refer to carcinomas. In embodiments, “epithelial-based cancer” can refer to adenocarcinomas. Non-limiting examples of an epithelial-based cancer include non-small cell lung cancer, prostate cancer, pancreatic cancer, esophageal cancer, colorectal cancer, breast cancer, stomach cancer, and ovarian cancer.

In embodiments, the cancer can comprise a pediatric cancer. The term “pediatric cancer” or “childhood cancer” as used herein can refer to any cancer occurring in a subject between birth and 19 years of age. In embodiments, “pediatric cancer” can refer to cancers occurring in a subject between birth and 14 years of age.

Exemplary pediatric cancers include acute lymphoblastic leukemia tumors, soft tissue sarcomas (e.g., rhabdomyosarcoma), rhabdoid tumors, neuroblastoma, kidney tumors, lymphoma, and brain and central nervous system tumors (e.g., astrocytoma, brain stem glioma, ependymoma, germ cell tumor, medulloblastoma). In aspects of the disclosure, the pediatric cancer is a rhabdoid tumor, a neuroblastoma, an acute lymphoblastic leukemia tumor, or a brain and central nervous system tumor.

The term “administration” or “administering” can refer to introducing a short-chain fatty acid or pharmaceutical composition comprising the same as described herein into a subject. For example, routes of administration can include orally, parenterally, topically, and intrathecally. However, any route of administration, such as subcutaneous, peritoneal, intra-arterial, inhalation, intranasal, parenteral, intravitreal, intraocular, ocular, subretinal, intrathecal, intravenous, subcutaneous, transcutaneous, intracutaneous, intracranial and the like administration. vaginal, rectal, nasal, or instillation into body compartments can be used. For example, “parenteral administration” can refer to administration via injection or infusion. Parenteral administration includes, but is not limited to, subcutaneous administration, intravenous administration, and intramuscular administration.

“Oral administration” can refer to a route of administration through the oral cavity or mouth. In embodiments, the short-chain fatty acid or pharmaceutical composition can be formulated to be ingested by the subject. “Ingested” can refer to the action of taking a substance into the body by swallowing. In further embodiments, the short-chain fatty acid or pharmaceutical composition can be provided as a tablet, a capsule, or a pre-made suspension. A “tablet” can refer to an entity that comprises active substances and excipients, for example in powder form, pressed or compacted from a powder into a solid dose. A “capsule” can refer to a soft gel, caplet, or any other encapsulated dosage form known to practitioners in the art. “Suspension” can refer to a heterogeneous mixture of a solid and liquid.

For oral preparations, the short-chain fatty acid or pharmaceutical composition can be used alone or in combination with appropriate additives to make tablets, powders, granules or capsules, for example, with conventional additives, such as lactose, mannitol, corn starch or potato starch; with binders, such as crystalline cellulose, cellulose derivatives, acacia, corn starch or gelatins; with disintegrators, such as corn starch, potato starch or sodium carboxymethylcellulose; with lubricants, such as talc or magnesium stearate; and if desired, with diluents, buffering agents, moistening agents, preservatives and flavoring agents.

For example, oral administration of a short-chain fatty acid or pharmaceutical composition comprising the same can be by way of a dietary supplement. The term “dietary supplement” can refer to a small amount of a composition for supplementation of a human or animal diet packaged in single or multiple dose units. Dietary supplements do not generally provide significant amounts of calories but can contain other micronutrients (e.g. vitamins or minerals). Dietary supplements can be in the form of a capsule, tablet, chewable soft gel, hard gelatine capsule, syrup, or elixir.

“Parenteral administration” can refer to administration via injection or infusion. Parenteral administration includes, but is not limited to, intraperitoneal administration, subcutaneous administration, intravenous administration, intramuscular administration.

“Topical administration” can refer to administration onto any accessible body surface of any human or animal species, such as the human species, for example, the skin or mucosal epithelia. In embodiments of this invention, “topical” can refer to an external application to the skin epithelium.

The term “intrathecal administration” can refer to the route of administration for a drug through injection into the spinal canal, more specifically into the subarachnoid space, so that it reaches cerebrospinal fluid.

In embodiments, the short-chain fatty acid or pharmaceutical composition comprising the same can be administered systemically. The term “systemically” can refer to the effect manifested in the area remote from the place of application and includes the entire body through which the circulatory system can carry it.

In embodiments, the short-chain fatty acid or pharmaceutical composition comprising the same can be administered intratumorally. As used herein, the term “intratumoral” or “intratumorally” can refer to administration directly into a tumor by injection, or delivery into an existing cavity in the body or cavity formed because of surgical resection of all or a portion of a solid tumor.

Described herein are methods and compositions for the administration of the active agent(s) to a subject using any available method and route suitable for drug delivery, including in vivo, in vitro and ex vivo methods, as well as systemic and localized routes of administration. Routes of administration include intranasal, intramuscular, intratracheal, subcutaneous, intra cerebroventricular, intradermal, topical application, intravenous, rectal, nasal, oral, and other enteral and parenteral routes of administration. Routes of administration can be combined, if desired, or adjusted depending upon the agent and/or the desired effect. An active agent can be administered in a single dose or in multiple doses.

In embodiments, the method can comprise administering to a subject a therapeutically effective amount of a short-chain fatty acid or pharmaceutical composition comprising the same. A “therapeutically effective amount” can refer to that amount which provides a therapeutic effect for a given condition and administration regimen. For example, “therapeutically effective amount” can refer to an amount that is effective to prevent, alleviate or ameliorate symptoms of the disease or prolong the survival of the subject being treated, which may be a human or non-human animal. Determination of a therapeutically effective amount is within the skill of the person skilled in the art.

In embodiments, a therapeutically effective amount of a short-chain fatty acid or pharmaceutical composition comprising the same can comprise less than about 0.1 mg/kg, about 0.1 mg/kg, about 0.5 mg/kg, about 1.0 mg/kg, about 2.5 mg/kg, about 5 mg/kg, about 7.5 mg/kg, about 10 mg/kg, about 15 mg/kg, about 20 mg/kg, about 25 mg/kg, about 30 mg/kg, about 35 mg/kg, about 40 mg/kg, about 45 mg/kg, about 50 mg/kg, about 55 mg/kg, about 60 mg/kg, about 70 mg/kg, about 80 mg/kg, about 90 mg/kg, about 100 mg/kg, about 120 mg/kg, about 135 mg/kg, about 150 mg/kg, about 175 mg/kg, about 200 mg/kg, about 225 mg/kg, about 250 mg/kg, about 275 mg/kg, about 300 mg/kg, about 325 mg/kg, about 350 mg/kg, about 375 mg/kg, about 400 mg/kg, about 425 mg/kg, about 450 mg/kg, about 475 mg/kg, about 500 mg/kg, about 525 mg/kg, about 550 mg/kg, about 575 mg/kg, about 600 mg/kg, about 625 mg/kg, about 650 mg/kg, about 675 mg/kg, about 700 mg/kg, about 725 mg/kg, about 750 mg/kg, about 775 mg/kg, about 800 mg/kg, about 825 mg/kg, about 850 mg/kg, about 875 mg/kg, about 900 mg/kg, about 1000 mg/kg, about 1500 mg/kg, about 2000 mg/kg, about 2500 mg/kg, about 5000 mg/kg, about 10000 mg/kg, about 25000 mg/kg, about 50000 mg/kg, or more than 50000 mg/kg of compound per body weight of a subject. For example, a therapeutically effective amount of a short-chain fatty acid or pharmaceutical composition can comprise about 2,000 mg/kg up to 10,000 mg/kg to treat a variety of cancers, such as those described herein.

In embodiments, the therapeutically effective amount of a short-chain fatty acid or pharmaceutical composition comprising the same comprises less than about 0.1 mg, about 0.1 mg, about 0.5 mg, about 1.0 mg, about 2.5 mg, about 5 mg, about 7.5 mg, about 10 mg, about 15 mg, about 20 mg, about 25 mg, about 30 mg, about 35 mg, about 40 mg, about 45 mg, about 50 mg, about 55 mg, about 60 mg, about 70 mg, about 80 mg, about 90 mg, about 100 mg, about 120 mg, about 135 mg, about 150 mg, about 175 mg, about 200 mg, about 225 mg, about 250 mg, about 275 mg, about 300 mg, about 325 mg, about 350 mg, about 375 mg, about 400 mg, about 425 mg, about 450 mg, about 475 mg, about 500 mg, about 525 mg, about 550 mg, about 575 mg, about 600 mg, about 625 mg, about 650 mg, about 675 mg, about 700 mg, about 725 mg, about 750 mg, about 775 mg, about 800 mg, about 825 mg, about 850 mg, about 875 mg, about 900 mg, about 1000 mg, about 1500 mg, about 2000 mg, about 2500 mg, about 5000 mg, about 10000 mg, about 25000 mg, about 50000 mg, or more than 50000 mg.

In embodiments comprising two or more short-chain fatty acids, the therapeutically effective amount can comprise any amount of the short-chain fatty acids that adds up to 100 wt % of the composition. For example, therapeutically effective amount can comprise any amount of a first short-chain fatty acid, any amount of a second short-chain fatty acid, any amount of a third short-chain fatty acid, and so on, such that the total adds up to 100 wt % of the total of the composition. Referring to FIGS. 2-8, for example, the combination composition can comprise 60% sodium acetate, 25% sodium propionate and 15% sodium butyrate. In embodiments, the mixture of two or more short-chain fatty acids can be provided as a ratio. Referring to FIGS. 2-8, for example, the combination composition can comprise a ratio of 6:2.5:1.5 of sodium acetate, sodium propionate, and sodium butyrate, respectively.

A therapeutically effective amount of a short-chain fatty acid or pharmaceutical composition can vary, depending on, for example, the age and weight of the subject, the cancer to be treated, the route of administration, and the concentration and/or formulation administered.

Embodiments can be administered to a subject in one or more doses. The dose level can vary as a function of the specific composition or pharmaceutical composition administered, the severity of the symptoms and the susceptibility of the subject to side effects. Dosages for a given compound are readily determinable by a variety of means. For example, dosages can be determined by standard clinical techniques. In addition, in vitro or in vivo assays can be employed to help identify optimal dosage ranges. The precise dose to be employed can also depend on the route of administration and can be decided according to the judgment of the practitioner and each patient's circumstances.

Unit dosage forms for oral administration, such as syrups, elixirs, and suspensions, can be provided wherein each dosage unit, for example, teaspoonful, tablespoonful, tablet, or suppository, contains a predetermined amount of the composition containing one or more compositions. Similarly, unit dosage forms for injection or intravenous administration may comprise the composition or pharmaceutical composition in a composition as a solution in sterile water, normal saline, or another pharmaceutically acceptable carrier.

In embodiments, multiple doses of the short-chain fatty acid or pharmaceutical composition comprising the same can be administered. The frequency of administration of the short-chain fatty acid or pharmaceutical composition can vary depending on any of a variety of factors, e.g., severity of the symptoms, and the like. For example, in an embodiment, the short-chain fatty acid or pharmaceutical composition can be administered once per month, twice per month, three times per month, every other week (qow), once per week (qw), twice per week (biw), three times per week (tiw), four times per week, five times per week, six times per week, every other day (qod), daily (ad), twice a day (qid), three times a day (tid), or four times a day. As discussed above, in an embodiment, the short-chain fatty acid or pharmaceutical composition can be administered for one day, five days, one week, one month, or longer than one month. In embodiments, the short-chain fatty acid or pharmaceutical composition can be administered for cycles of five consecutive days over a period of one week, one month, one year, or longer than one year.

In embodiments, the short-chain fatty acid or pharmaceutical composition comprising the same can be administered in combination with one or more additional active agents. For example, a first agent (e.g., a prophylactic or therapeutic agent) can be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second agent (e.g., a prophylactic or therapeutic agent) to a subject with a disease or disorder or a symptom thereof.

The duration of administration of the short-chain fatty acid or pharmaceutical composition, e.g., the period of time over which the short-chain fatty acid or pharmaceutical composition is administered, can vary, depending on any of a variety of factors, including patient response. In vitro or in vivo assays can be employed to help identify optimal dosage ranges. The precise dose to be employed can also depend on the route of administration and can be decided according to the judgment of the practitioner and each patient's circumstances.

In pharmaceutical dosage forms, the short-chain fatty acid or pharmaceutical composition comprising the same can be administered in the form of its pharmaceutically acceptable salts. A pharmaceutically active composition can be used alone or in appropriate association, as well as in combination, with other pharmaceutically active compounds.

In embodiments, the short-chain fatty acid or pharmaceutical composition comprising the same can be administered as part of a therapeutic regimen. A “treatment regimen” or “therapeutic regimen” can refer to regimen carried out on a patient for the purposes of treatment, as described above, e.g., for slowing, stopping or reversing the disease's progression, as evidenced by decreasing, cessation or elimination of either clinical or diagnostic symptoms. A treatment regimen can be performed to reduce disease severity, improve and stabilize the disease symptoms of the patient.

In embodiments, the short-chain fatty acid or pharmaceutical composition comprising the same can be administered to a subject in combination with one or more additional anti-cancer therapies. For example, the one or more additional anti-cancer therapies can include but is not limited to chemotherapy, immunotherapy, radiotherapy, surgical therapy, or a combination of the foregoing and/or other therapies useful in the prevention, management and/or treatment of cancer or one or more symptoms thereof.

In embodiments, the short-chain fatty acid and a second active agent can be administered sequentially, such as one before the other, or concurrently or simultaneously, such as at about the same time. For example, a second active agent can include but is not limited to another anti-cancer agent, an anti-nausea agent, an anti-inflammatory agent, a radioactive agent, a heavy metal, or a combination of the foregoing and/or other agents useful in the prevention, management and/or treatment of cancer or one or more symptoms thereof.

For example, an anti-cancer agent can include a chemotherapeutic or a growth inhibitory agent, including but not limited to an alkylating agent, an anthracycline, an anti-hormonal agent, an aromatase inhibitor, an anti-androgen, a protein kinase inhibitor, a lipid kinase inhibitor, an antisense oligonucleotide, a ribozyme, an antimetabolite, a topoisomerase inhibitor, a cytotoxic agent or antitumor antibiotic, a proteasome inhibitor, an anti-microtubule agent, an EGFR antagonist, a retinoid, a tyrosine kinase inhibitor, a histone deacetylase inhibitor, and combinations thereof.

The term “anti-nausea agent” can refer to an agent that has an anti-nausea or anti-emetic effect. “Anti-nausea agent” can refer to an agent that (1) prevents nausea or vomiting from occurring in a subject who may be predisposed to the nausea or vomiting; (2) inhibits nausea or vomiting in a subject who may be experiencing nausea or vomiting; or (3) ameliorates or relieves the symptoms of the nausea or vomiting. For example, an anti-nausea agent can include dimenhydrinate (dramamine), domperidone, scopolamine (hyoscine), cinnarizine, metoclopramide, cyclizine, and promethazine.

The term “anti-inflammatory agent” can refer to a non-steroidal anti-inflammatory drug (“NSAID”). For example, an anti-inflammatory agent can include acetaminophen, amoxiprin, benolylate, choline magnesium salicylate, difunisal, faislamine, methyl salicylate, magnesium salicylate, salicylate, diclofenac, aceclofenac, acemethacin, bromfenacmetrod, Sulindac, tolmethine, ibuprofen, carprofen, fenbufen, fenoprofen, flurbiprofen, ketoprofen, loxoprofen, naproxen, thiaprofenic acid, mefenamic acid, meclofenamic acid, tolfenamic acid, phenylbutazone, azapropazone, metamizole, oxyphenbutazone, Piroxicam, lornoxicam, meloxy Beam, a tenoxicam, celecoxib, etoricoxib, lumiracoxib, parecoxib, nimesulide, licofelone, indomethacin, COX-2 inhibitors or pharmaceutically acceptable salts and mixtures thereof.

The term “radioactive agent” can refer to a substance (for example, a radionuclide or radioisotope) which loses energy (for example, decays) by emitting ionizing particles and radiation. When the substance decays, its presence can be determined by detecting the radiation emitted by it. Non-limiting examples of radioactive agents include ^{99m}Tc, ¹⁸F, ¹³¹I and ¹²⁵I. For example, a radioactive agent can include a heavy metal.

The term “simultaneous administration” can refer to a first agent and a second agent, when together in the therapeutic combination therapy, are administered either less than about 15 minutes, e.g., less than about 10, 5, or 1 minute. When the first agent and the second agent are administered simultaneously, the first and second treatments can be in the same composition (e.g., a composition comprising both the first and second therapeutic agents) or separately (e.g., the first therapeutic agent is contained in one composition and the second treatment is contained in another composition).

The term “sequential administration” can refer to a first agent and a second agent administered to a subject greater than about 15 minutes apart, such as greater than about 20, 30, 40, 50, 60 minutes, or greater than 60 minutes apart. Either agent can be administered first. For example, the first agent and the second agent can be included in separate compositions, which can be included in the same or different packages or kits.

The terms “co-administration” or the like, as used herein, can refer to the administration of a first active agent and at least one additional active agent to a single subject, and is intended to include treatment regimens in which the compounds and/or agents are administered by the same or different route of administration, in the same or a different dosage form, and at the same or different time.

The term “in combination” can refer to the use of more than one therapies (e.g., one or more prophylactic and/or therapeutic agents). The use of the term “in combination” does not restrict the order in which therapies are administered to a subject with a disease or disorder, or the route of administration.

Aspects of the invention are directed towards kits, such as kits comprising compositions as described herein for treating or preventing cancer. For example, the kit can comprise therapeutic combination compositions described herein.

In one embodiment, the kit includes (a) a container that contains an anti-cancer composition, such as that described herein, and optionally (b) informational material. The informational material can be descriptive, instructional, marketing, or other material that relates to the methods described herein and/or the use of the agents for therapeutic benefit.

The informational material of the kits is not limited in its form. In one embodiment, the informational material can include information about production of the compound, molecular weight of the compound, concentration, date of expiration, batch, or production site information, and so forth. In one embodiment, the informational material relates to methods of administering the therapeutic combination composition, e.g., in a suitable dose, dosage form, or mode of administration (e.g., a dose, dosage form, or mode of administration described herein), to treat a subject who has a nerve disconnectivity disorder). The information can be provided in a variety of formats, include printed text, computer readable material, video recording, or audio recording, or information that provides a link or address to substantive material.

“Instructional material,” as that term is used herein, includes a publication, a recording, a diagram, or any other medium of expression which can be used to communicate the usefulness of components of the invention in the kit for identifying or alleviating or treating the various diseases or disorders recited herein. Optionally, or alternately, the instructional material may describe one or more methods of identifying or alleviating the diseases or disorders in a cell or a tissue of a subject. The instructional material of the kit may, for example, be affixed to a container that contains the compositions of the invention or be shipped together with a container that contains the compositions of the invention. Alternatively, the instructional material may be shipped separately from the container with the intention that the recipient uses the instructional material and the compound cooperatively.

The composition in the kit can include other ingredients, such as a solvent or buffer, a stabilizer, or a preservative. The antagonist can be provided in any form, e.g., liquid, dried or lyophilized form, such as substantially pure and/or sterile. When the agents are provided in a liquid solution, the liquid solution, for example, is an aqueous solution. When the agents are provided as a dried form, reconstitution generally is by the addition of a suitable solvent. The solvent, e.g., sterile water or buffer, can optionally be provided in the kit.

The kit can include one or more containers for the composition or compositions containing the agents. In some embodiments, the kit contains separate containers, dividers or compartments for the composition and informational material. For example, the composition can be contained in a bottle, vial, or syringe, and the informational material can be contained in a plastic sleeve or packet. In other embodiments, the separate elements of the kit are contained within a single, undivided container. For example, the composition is contained in a bottle, vial or syringe that has attached thereto the informational material in the form of a label. In some embodiments, the kit includes a plurality (e.g., a pack) of individual containers, each containing one or more unit dosage forms (e.g., a dosage form described herein) of the agents. The containers can include a combination unit dosage, e.g., in a desired ratio. For example, the kit includes a plurality of syringes, ampules, foil packets, blister packs, or medical devices, e.g., each containing a single combination unit dose. The containers of the kits can be airtight, waterproof (e.g., impermeable to changes in moisture or evaporation), and/or light tight. The kit optionally includes a device suitable for administration of the composition, e.g., a syringe or other suitable delivery device. The device can be provided pre-loaded with one or both agents or can be empty, but suitable for loading.

While the invention has been described in conjunction with the detailed description thereof, the foregoing description is intended to illustrate and not limit the scope of the invention, which is defined by the scope of the appended claims. Other compositions, compounds, methods, features, and advantages of the disclosure will be or become apparent to one having ordinary skill in the art upon examination of the following drawings, detailed description, and examples. It is intended that all such additional compositions, compounds, methods, features, and advantages be included within this description, and be within the scope of the disclosure.

The invention will be further described in the following examples, which do not limit the scope of the invention described in the claims.

### EXAMPLES

Examples are provided below to facilitate a more complete understanding of the invention. The following examples illustrate the exemplary modes of making and practicing the invention. However, the scope of the invention is not limited to specific embodiments disclosed in these Examples, which are for purposes of illustration only, since alternative methods can be utilized to obtain similar results.

### Example 1

### Example 1—Efficacy Study with Short-Chain Fatty Acids in an In Vivo Hollow Fiber Model Using the Human Pancreatic Adenocarcinoma BxPC-3, Colon Adenocarcinoma Caco-2 and Lung Non-Small Cell Lung Cancer H460 Cell Lines in Female NMRI Nude Mice

### Aim of the Study

In this in vivo study, the short-chain fatty acids sodium acetate, sodium propionate and sodium butyrate were evaluated in an efficacy study with respect to their anti-tumoral efficacy in human pancreatic adenocarcinoma BxPC-3, colon adenocarcinoma Caco-2 and lung non-small cell lung cancer H460 cell lines using the Hollow Fiber model in female NMRI nude mice.

### SUMMARY

The study consisted of 2 groups, each containing 6 female NMRI nude mice after randomization. On Day 0, three different Hollow Fibers (distinguishable from each other by color or marked margins), which had been loaded 24 h prior to implantation with 8.0×106/ml BxPC-3 cells, 8.0×106/ml Caco-2 cells or 3.0×106/ml H460 cells, were implanted both subcutaneously as well as intraperitoneally into all animals.

Starting on Day 1, after animals had been randomized according to body weights, treatment was initiated for all groups. Animals of Group 1 were treated with 5 ml/kg Vehicle i.p. five days a week on Days 2-6 and 9-13 and animals of Group 2 were treated with 2000 mg/kg (5 ml/kg) short-chain fatty acids sodium acetate, sodium propionate and sodium butyrate (SCFA) i.p. five days a week on Days 2-6 and 9-13. No animal was found dead or was euthanized prior to the study end due to ethical abortion criteria. On Day 16, all study groups were terminated, all respective animals euthanized, and a necropsy performed.

During necropsy, the implanted Hollow Fibers were collected, appropriately processed and a CellTiter Glo® assay for assessing cell viability performed (for details, please refer also to section 6 with the raw data).

In summary, according to CTG assay, for intraperitoneal implantation all three cell lines showed anti-tumoral effects with the best and significant result for Hollow Fibers loaded with Caco-2 cells. In case of subcutaneous implantation, a slight signal decrease was detected for Hollow Fibers loaded with H460 cells, no signal differences were observed for Hollow Fibers loaded with BxPC-3 or Caco-2 cells.

Only for Hollow Fibers loaded with H460 cells a noticeable signal reduction was detected in both compartments s.c. and i.p. indication a probably inhibitory effect in a subsequent xenograft study. For BxPC-3 and Caco-2, signal reductions were only detected for intraperitoneal implantation indicating optimization of the compound to be effective in a subsequent xenograft study.

### Detailed Findings

### Animal Weights

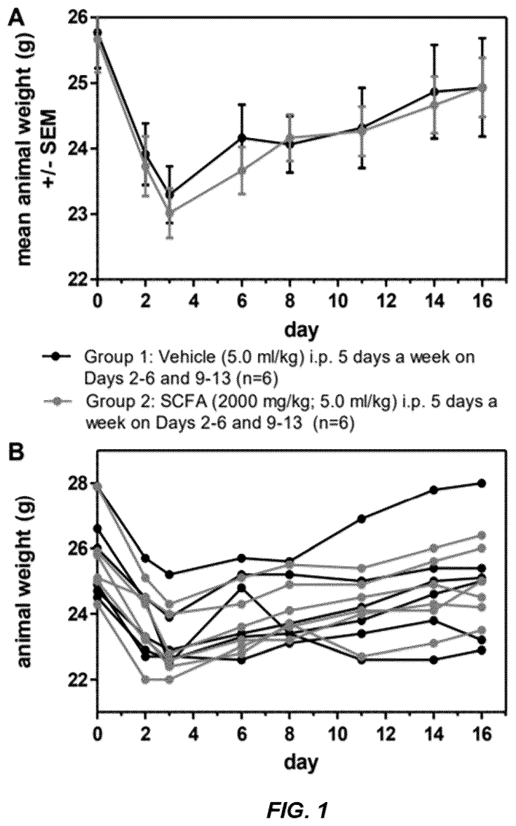

Following Hollow Fiber implantation on Day 0, animal weights of all study groups first noticeably decreased, probably due to induced stress during surgery (FIG. 1). Treatment was initiated on Day 1. Starting on Day 3, animal weights continuously increased during the treatment period until study end.

### CellTiter Glo Assay

In order to assess cell viability, implanted Hollow Fibers were excised, containing cells appropriately processed and a CellTiter Glo® assay performed (FIGS. 2-4).

### BxPC-3

Intraperitoneal implantation of Hollow Fibers loaded with BxPC-3 tumor cells resulted in a significant anti-tumoral effect (38% here as in the following of corresponding vehicle control). In contrast, subcutaneous implantation showed no effect (98%).

### Caco-2

Intraperitoneal implantation of Hollow Fibers loaded with Caco-2 tumor cells showed a slight signal decrease (80%), subcutaneous implantation showed no effect (100%).

### H460

Intraperitoneal as well as subcutaneous implantation of Hollow Fibers loaded with H460 tumor cells resulted in slight anti-tumoral effects without significance (79 and 71%, respectively).

### Animal Welfare

This animal study has been approved by the Ethics Committee for Animal Experimentation and is registered by the regional board Freiburg. Mice are handled according to the German animal welfare law and the GV-SOLAS guidelines. Health monitoring of the animal facility is done according to FELASA guidelines quarterly by examination of sentinel animals.

In general, mice delivered for this study were maintained in individually ventilated cages at constant temperature and humidity. Animal behavior was monitored daily throughout the study. After start of therapy, animal weights were determined. Deviation of the health status of the animals were documented and animals were euthanized individually before study termination when ethical abortion criteria were reached (e.g. body weight loss≥20%, signs of sickness, ascites).

### Materials and Methods

### Test System

### Test Animals

Species: Mus musculus
Strain: NMRI nude (Crl: NMRI-Foxn1nu)
Sex: Female
Source: Charles River GmbH Sandhofer Weg 7 97633 Sulzfeld Germany
Number of animals: 12
Age at delivery: 5-6 weeks
Identification: Labeling by tattoo
Acclimatization: March 22nd to 24th, 2021

### Cell Culture

Cell line: BxPC-3
CPQ (PQ identifier): #291
Origin: Originated from human pancreatic adenocarcinoma
Modification: None
Incubation: 37° C., 5% CO2
Medium: RPMI 1640 high Glutamax 1 with 10% FCS, 100 units penicillin/ml, and 100 μg of streptomycin/ml
Expansion: 70%-90% confluent cultures were split routinely using accutase
Quality control: Routine cell line authentication by a third party, as well as inhouse Mycoplasma testing using PCR
Cell line: Caco-2
CPQ (PQ identifier): #27
Origin: Originated from human colon adenocarcinoma
Modification: None
Incubation: 37° C., 10% CO2
Medium: DMEM Glutamax+4.5 g/L Glucose with 10% FCS, 100 units penicillin/ml, and 100 μg of streptomycin/ml
Expansion: 70%-90% confluent cultures were split routinely using trypsin/EDTA
Quality control: Routine cell line authentication by a third party, as well as inhouse mycoplasma testing using PCR
Cell line: H460
CPQ (PQ identifier): #48
Origin: Originated from human lung non-small cell lung cancer
Modification: None
Incubation: 37° C., 10% CO2
Medium: DMEM Glutamax+4.5 g/L Glucose with 10% FCS, 100 units penicillin/ml, and 100 μg of streptomycin/ml
Expansion: 70%-90% confluent cultures were split routinely using trypsin/EDTA
Quality control: Routine cell line authentication by a third party, as well as inhouse Mycoplasma testing using PCR

TABLE 2
Material List

| | | Catalogue | | |
|---|---|---|---|---|
| Item | Manufacturer | number | Lot number | Expiry date |
| RPMI 1640 high | Gibco by | 61870-010 | 2293657 | February 2022 |
| Glutamax 1 | life techn. | | | |
| DMEM Glutamax + | Gibco by | 61965-026 | 0593655 | February 2022 |
| 4.5 g/L Glucose | life techn. | | | |
| PBS | Capricorn | PBS-1A | CP20-3612 | July 2023 |
| FCS | Bio Sell | FCS.SAM.0500 | BS240921 | June 2022 |
| penicillin/ | Capricorn | PS-B | CP20-3341 | June 2022 |
| streptomycin | | | | |
| accutase | Capricorn | ACC-1B | CP21-4166 | November 2022 |
| trypsin/EDTA | Capricorn | TRY-1B | CP20-3394 | March 2022 |

### Husbandry

Room number: EG-05
Conditions: Optimum hygienic conditions, air-conditioned with 10-15 air changes per hour, and continually monitored environment with target ranges for temperature 22±2° C. and for relative humidity 45-65%, 12 hours artificial fluorescent lighting/12 hours darkness.
Accommodation: max. 4 animals per individual ventilated cage (IVC, Zoonlab; Castrop-Rauxel, Germany; max. 4 mice/cage; cage size: 38×16×13 cm), at randomization mice assigned to the same group are housed together
Diet: M-Zucht (#V1126) ssniff Spezialdiäten GmbH Ferdinand-Gabriel-Weg 16 59494 Soest, Germany
Water: Community tap water (autoclaved)

### Tumor Cell Loading of Hollow Fibers

On Day-1, tumor cells were loaded into the Hollow Fiber, placed in cell culture dishes containing RPMI-1640 medium with 20% FCS and 1% Penicillin/Streptomycin, and were equilibrated in the incubator at 37° C., 5% CO2 overnight. BxPC-3 tumor cells were loaded with a cell number of 8.0×106/ml using RPMI-1640 medium with 20% FCS and 1% Penicillin/Streptomycin into KrosFlow hollow fiber membranes (white fibers). Caco-2 tumor cells were loaded with a cell number of 8.0×106/ml using RPMI-1640 medium with 20% FCS and 1% Penicillin/Streptomycin into KrosFlow hollow fiber membranes (white fibers with margin marks). H460 tumor cells were loaded with a cell number of 3.0×106/ml using RPMI-1640 medium with 20% FCS and 1% Penicillin/Streptomycin into KrosFlow hollow fiber membranes (blue fibers).

### Hollow Fiber Implantation

On Day 0, 1-2 hours before surgery mice received the analgeticum Meloxicam in 0.9% NaCl (1.0 mg/kg in 10.0 ml/kg) subcutaneously with repetition 24 hours later. Implantation was performed while mice were under inhalational isoflurane anesthesia. Three fibers were implanted into two different compartments: subcutaneous and intraperitoneal. Therefore, every mouse received 6 fibers. In detail, a trocar containing the fibers was inserted through a skin incision to place fibers subcutaneously and peritoneally using the same skin incision with further perforating the peritoneum. Skin was closed using suture clips.

### Observations and Data Recording

Animal monitoring occurred daily and included inspection and documentation of abnormalities. Clinical signs were inspected daily, and documentation of abnormalities occurred daily. Animal body weights were recorded three times a week after the start of therapy.

### Study Design

### Randomization

On Day 2 after surgery, mice were block-randomized according to animal weights. For block randomization, a robust automated random number generation within individual blocks was used (MS-Excel 2016).

### Treatment

Mice were treated according to the following dosing schedule. Treatment started on Day 2, the day of randomization.

TABLE 3
Dosing schedule

| | | | Dose | | | | |
|---|---|---|---|---|---|---|---|
| | Test | Dose | volume | Administration | Dosing | | Number of |
| Group | specimen | (mg/kg) | (ml/kg) | route | frequency | Animal ID¹⁾ | animals |
| 1 | Vehicle | — | 5 | i.p. | 5 days a | 2, 5, 7, 9, 10, 11 | 6 |
| | | | | | week on | | |
| | | | | | Days 2-6 | | |
| | | | | | and 9-13 | | |
| 2 | short-chain fatty | 2000 | 5 | i.p. | 5 days a | 1, 3, 4, 6, 8, 12 | 6 |
| | acids | | | | week on | | |
| | | | | | Days 2-6 | | |
| | | | | | and 9-13 | | |

¹⁾All animals listed here share the common suffix/21

### Termination

No individual animals were euthanized due to ethical abortion criteria or found dead prior to study end without performing a necropsy. On Day 16, the study was terminated, all animals were sacrificed, and a necropsy was performed.

### Necropsy

On Day 16, 14 days after therapy, animals were euthanized by cervical dislocation and fibers extracted. Each fiber was processed according to the Cell-Titer-Glo assay protocol.

### CellTiter Glo Assay

Each fiber was cut into 4 pieces and smashed using the Smasher Fast Prep24 system. The supernatant was incubated with CellTiter Glo buffer for 10 min in the dark while shaking and luminescence was detected with an Enspire/EnVision plate reader (Perkin Elmer).

### Statistical Analysis

All numerical data of this study are graphically displayed in the final study report and additionally listed as tables in section 6. Data of the individual groups were analyzed using descriptive data analysis (Mean with SEM, Median and interquartile range). Statistical analysis of efficacy data was done using the Mann Whitney test and the unpaired Student's t-test. All data analysis was performed using GraphPad Prism 5 from GraphPad Software, Inc., San Diego, USA.

### Test Specimen

### Substance Information

TABLE 4
Substance information

| | | | | | | Date of |
|---|---|---|---|---|---|---|
| | | | Catalogue | Lot | Delivery | compound |
| Substance | Provider | Manufacturer | number | number | date | disposal |
| Sodium | LSU HSC | Sigma-Aldrich | S2889 | SLCF4431 | Jan. 3, 2021 | no |
| acetate | | | | | | |
| Sodium | LSU HSC | Sigma-Aldrich | P1880 | SLCF2447 | Jan. 3, 2021 | no |
| propionate | | | | | | |
| Sodium | LSU HSC | Sigma-Aldrich | B5887 | SLBQ8041V | Jan. 3, 2021 | no |
| butyrate | | | | | | |

### Substance Handling

Reaction Biology handled submitted compound(s) according to the Material Safety Data Sheet(s) (relevant MSDS(s) which was provided together with the test compound(s)).

### Vehicle Information

TABLE 5
Vehicle Information

| | | | Catalog | | Expiration |
|---|---|---|---|---|---|
| Substance | Provider | Manufacturer | number | Lot number | date |
| Aqua ad | Reaction | Braun | PZN6724092 | 204938081 | November 2023 |
| injectabilia | Biology | | | | |

### Test Specimen Formulation

TABLE 6
Test specimen formulation

| | | Formulation | Final | Frequency of |
|---|---|---|---|---|
| Test | Vehicle | preparation | compound | formulation |
| specimen | composition | procedure | appearance | preparation |
| Vehicle | water | — | Clear | Once weekly |
| | | | solution | |
| Short Chain | water | solubilized 60% acetate: 25% | Clear | Once weekly |
| Fatty Acids | | propionate 15% N-butyrate in | solution | |
| | | water | | |

### Test Specimen Storage

TABLE 7
Test specimen storage

| | | | Working | Working | Working |
|---|---|---|---|---|---|
| | Stock | Stock | solution | solution | solution |
| Test | concentration | storage | conc. | storage | stability |
| specimen | (mg/ml) | (° C.) | (mg/ml) | (° C.) | (d) |
| Vehicle | — | RT | — | — | — |
| Short | 2000 | RT | 400 | 4° C. | >7 |
| Chain | | | | | |
| Fatty | | | | | |
| Acids | | | | | |

### Results

See, FIGS. 1 to 4.

### Raw Data

### Animal Weight

TABLE 8A
Animal weight (g)
Group 1: Vehicle (5 ml/kg) i.p. 5 days a week on Days 2-6 and 9-13

| ID. No.¹⁾ | 2 | 5 | 7 | 9 | 10 | 11 | Mean | SEM |
|---|---|---|---|---|---|---|---|---|
| Day 0 | 24.5 | 26.6 | 24.7 | 27.9 | 24.9 | 26.0 | 25.77 | 0.541 |
| 2 | 22.9 | 24.4 | 23.3 | 25.7 | 22.7 | 24.5 | 23.92 | 0.471 |
| 3 | 22.6 | 22.5 | 22.9 | 25.2 | 22.7 | 23.9 | 23.30 | 0.433 |
| 6 | 23.3 | 24.8 | 23.4 | 25.7 | 22.6 | 25.2 | 24.17 | 0.504 |
| 8 | 23.4 | 23.4 | 23.7 | 25.6 | 23.1 | 25.2 | 24.07 | 0.432 |
| 11 | 23.8 | 22.6 | 24.2 | 26.9 | 23.4 | 25.0 | 24.32 | 0.611 |
| 14 | 24.6 | 22.6 | 25.0 | 27.8 | 23.8 | 25.4 | 24.87 | 0.713 |
| 16 | 25.0 | 22.9 | 25.1 | 28.0 | 23.2 | 25.4 | 24.93 | 0.748 |

¹⁾All animal IDs listed here share the common suffix/21

TABLE 8B
Animal weight (g)
Group 2: SCFA (2000 mg/kg: 5 ml/kg) i.p. 5 days a
week on Days 2-6 and 9-13

| ID. No.¹⁾ | 1 | 3 | 4 | 6 | 8 | 12 | Mean | SEM |
|---|---|---|---|---|---|---|---|---|
| Day 0 | 25.9 | 25.8 | 27.9 | 24.3 | 25.0 | 25.1 | 25.67 | 0.506 |
| 2 | 24.3 | 23.3 | 25.1 | 22.0 | 23.2 | 24.5 | 23.73 | 0.457 |
| 3 | 22.8 | 22.4 | 24.3 | 22.0 | 22.6 | 24.0 | 23.02 | 0.376 |
| 6 | 23.6 | 22.8 | 25.1 | 23.0 | 23.2 | 24.3 | 23.67 | 0.359 |
| 8 | 24.1 | 23.7 | 25.5 | 23.6 | 23.2 | 24.9 | 24.17 | 0.356 |
| 11 | 24.5 | 22.7 | 25.4 | 24.1 | 24.0 | 24.9 | 24.27 | 0.378 |
| 14 | 24.9 | 23.1 | 26.0 | 24.1 | 24.3 | 25.6 | 24.67 | 0.433 |
| 16 | 24.5 | 23.5 | 26.4 | 25.0 | 24.2 | 26.0 | 24.93 | 0.450 |

¹⁾All animal IDs listed here share the common suffix/21

### CellTiter Glo Data

TABLE 9A
CellTiter Glo ® assay (photons/second) from BxPC-3-loaded Hollow
Fibers implanted both subcutaneously (s.c.) and intraperitoneally (i.p.) on Day 0.

| Group 1: Vehicle (5 ml/kg) i.p. 5 | | | Group 2: SCFA (2000 mg/kg; 5 ml/kg) | | |
|---|---|---|---|---|---|
| days a week on Days 2-6 and 9-13 | | | i.p. 5 days a week on Days 2-6 and 9-13 | | |
| ID. #¹⁾ | s.c. | i.p. | ID. # | s.c. | i.p. |
| 2 | 1910000 | 3170000 | 1 | 1990000 | 1710000 |
| 5 | 1900000 | 2600000 | 3 | 1780000 | 1790000 |
| 7 | 2250000 | 2800000 | 4 | 2120000 | 39500 |
| 9 | 1820000 | 2270000 | 6 | 1640000 | 323000 |
| 10 | 2160000 | 4280000 | 8 | 2570000 | 659000 |
| 11 | 3670000 | 2780000 | 12 | 1700000 | 2360000 |
| Mean | 2.285e+006 | 1.967e+006 | Mean | 2.983e+006 | 1.147e+006 |
| Std. Dev | 698591 | 346622 | Std. Dev. | 699562 | 932251 |
| Std. Error | 285199 | 141508 | Std. Error | 285595 | 380590 |

¹⁾All animal IDs listed here share the common suffix/21

TABLE 9B
CellTiter Glo ® assay (photons/second) from Caco-2-loaded Hollow
Fibers implanted both subcutaneously (s.c.) and intraperitoneally (i.p.) on Day 0.

| Group 1: Vehicle (5 ml/kg) i.p. 5 | | | Group 2: SCFA (2000 mg/kg; 5 ml/kg) | | |
|---|---|---|---|---|---|
| days a week on Days 2-6 and 9-13 | | | i.p. 5 days a week on Days 2-6 and 9-13 | | |
| ID. #¹⁾ | s.c. | i.p. | ID. # | s.c. | i.p. |
| 2 | 2120000 | 5070000 | 1 | 3910000 | 1500000 |
| 5 | 2220000 | 2940000 | 3 | 2150000 | 2590000 |
| 7 | 1400000 | 4230000 | 4 | 2800000 | 2870000 |
| 9 | 2080000 | 2040000 | 6 | 2200000 | 3310000 |
| 10 | 3410000 | 3340000 | 8 | 1280000 | 2000000 |
| 11 | 2800000 | 895000 | 12 | 1670000 | 2510000 |
| Mean | 2.338e−006 | 3.086e−006 | Mean | 2.335e+006 | 2.463e+006 |
| Std. Dev | 688547 | 1.498e+006 | Sid. Dev | 927852 | 639239 |
| Std. Error | 281098 | 611703 | Sid. Error | 378794 | 260968 |

¹⁾All animal IDs listed here share the common suffix/21

TABLE 9C
CellTiter Glo ® assay (photons/second) from H460-loaded Hollow
Fibers implanted both subcutaneously (s.c.) and intraperitoneally (i.p.) on Day 0.

| Group 1: Vehicle (5 ml/kg) i.p. 5 | | | Group 2: SCFA (2000 mg/kg; 5 ml/kg) | | |
|---|---|---|---|---|---|
| days a week on Days 2-6 and 9-13 | | | i.p. 5 days a week on Days 2-6 and 9-13 | | |
| ID. #¹⁾ | s.c. | i.p. | ID. # | s.c. | i.p. |
| 2 | 6840000 | 2930000 | 1 | 8590000 | 3200000 |
| 5 | 6020000 | 6050000 | 3 | 5970000 | 5240000 |
| 7 | 1.030e+007 | 8070000 | 4 | 4330000 | 6450000 |
| 9 | 2110000 | 6990000 | 6 | 4990000 | 4810000 |
| 10 | 4600000 | 6710000 | 8 | 4290000 | 1250000 |
| 11 | 7890000 | 8770000 | 12 | 1720000 | 7140000 |
| Mean | 6.293e+006 | 6.587e+006 | Mean | 4.982e+006 | 4.682e+006 |
| Std. Dev | 2.805e+006 | 2.040e+006 | Std. Dev | 2.260e+006 | 2.167e+006 |
| Std. Error | 1.145e+006 | 832753 | Std. Error | 922650 | 884501 |

¹⁾All animal IDs listed here share the common suffix/21

### Abbreviations

approx. approximately
BLI bioluminescence imaging
CD cluster of differentiation
CoA Certificate of analysis
d day
DMEM Dulbecco's Modified Eagle Medium
EDTA ethylenediaminetetraacetic acid
FCS fetal calf serum
FELASA Federation of Laboratory Animal Science Associations
FFPE formalin-fixed, paraffin-embedded
GV-SOLAS Gesellschaft für Versuchstierkunde/Society of Laboratory Animal Science
i.p. intraperitoneal
i.v. intravenous
IVC individually ventilated cage
i.t. intratumoral
i.ma. intramammary
max. maximal
min minute
MS Microsoft
n.a. not applicable
PCR polymerase chain reaction
PD pharmacodynamic
PK pharmacokinetic
PBS phosphate buffered saline
p.o. per os
rpm revolutions per minute
RPMI-1640 Roswell Park Memorial Institute-1640 medium
RT room temperature
S.C. subcutaneous
SEM standard error of mean
tbd to be determined

### Example 2

Many investigations seek to determine the link between gut microbiota and cancer
link can be subsequent to bacterial metabolites such as SCFA which are derivatives of fiber
established an enhanced rate of inflammatory disorders and cancer in subjects with diets poor in SCFA

### Mechanism of Action

Inhibit cell growth and migration
Suppress histone deacetylases (promotes tumorigenesis via gene transcription)
Induce apoptosis
Protects epithelial layer (anti-inflammatory via increase in tight junction, upregulates t-cell production)

### Anti-Tumor Effects

Colorectal Cancer
   Butyrate—Butyrate mediates suppression of NK-κB, inhibits HDAC, induces WNT/beta-catenin activity and apoptosis, activates MAPK signaling pathway by the upregulation of GADD153 or activation or phosphorylation of JNK
   Acetate—Acetate triggers apoptosis in colorectal cancer cells activates cathepsin D release and induces lysosomal membrane permeabilization. In return, cathepsin D protects colorectal cancer cells from apoptosis by disruption of damages mitochondria
   Propionate-Propionate reduces the expression of PRMT1, induces apoptosis in colon cancer by hindering P70 S6 kinase phosphorylation
Pancreatic Cancer
   Butyrate—Sodium butyrate hinders the expression of b4 Integrin, reduces the cell surface expression of b4, and hinders the invasion of pancreatic cancer
   Acetate—Medroxyprogesterone acetate blocks the proliferation of pancreatic carcinoma cell lines (Capan-2, AsPC-1, and MiaPaCa-2) and leads to cell detachment and reduces cell density
Lung Cancer
   Butyrate—Sodium butyrate increases the expression levels of P-gp and STATS3. It also increases STAT3 phosphorylation and improves mRNA stability of ABCB1 in human lung cancer cells
   Propionate acts as an anticancer component for lung cancer therapy, by triggering cell apoptosis and cell cycle arrest by up- and down-regulation of p21 and Survivin expression, respectively.

### Aims

Short-chain fatty acids sodium acetate, sodium propionate and sodium butyrate were evaluated for their anti-tumoral efficacy in human pancreatic adenocarcinoma, colon adenocarcinoma, non-small lung cancer
Pancreatic adenocarcinoma cell line: BxPC-3
Colon adenocarcinoma cell line: Caco-2
Non-small lung cancer: H460
Hollow Fiber model in female NMRI nude mice

### Study Design

2 groups each with 6 mice
   Group 1: 5 ml/kg vehicle 5 days a week (days 2-6, 9-13)
   Group 2: 5 ml/kg SCFA 5 days a week (days 2-6, 9-13)
Day 0:3 different Hollow Fibers loaded with BxPC-3, Caco-2, H460 cell lines implanted subcutaneously and intraperitoneally
Day 16: euthanized and necropsy preformed
   Hollow Fibers collected and cell titer glo (CTG) assessed cell viability

### CTG Analysis

| | | | Pancreas | | Colon | | Lung | |
|---|---|---|---|---|---|---|---|---|
| | | Dose | SQ | IP | SQ | IP | SQ | IP |
| Group | Specimen | (mg/kg) | (% of mean vehicle using CTG signal) | | | | | |
| 1 | Vehicle | — | 100 | 100 | 100 | 100 | 100 | 100 |
| 2 | SCFA | 2000 | 98 | 38 | 100 | 80 | 79 | 71 |

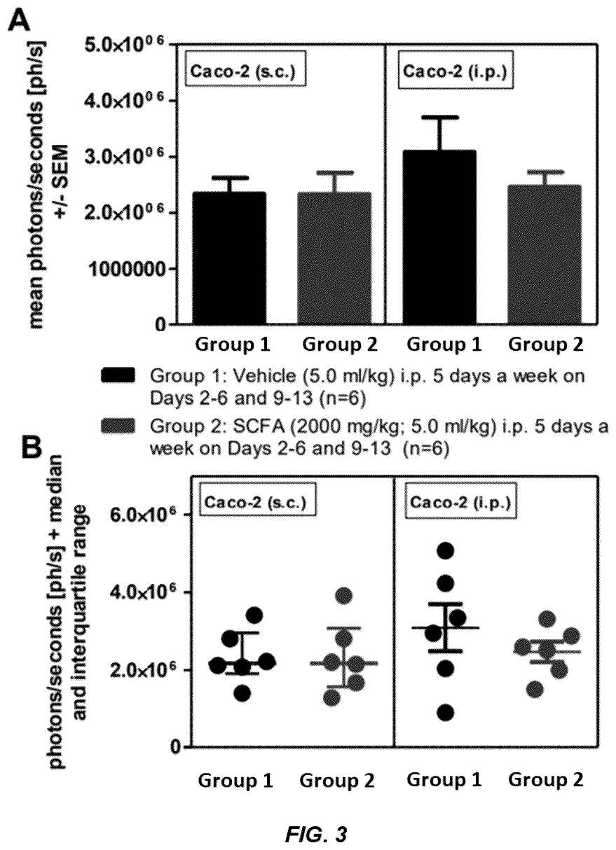

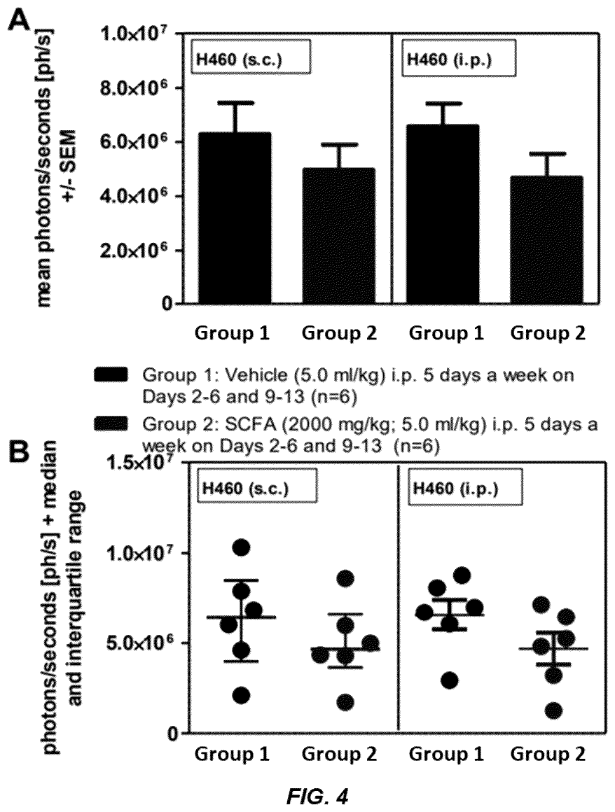

CTG Colon Cancer—see, for example, FIG. 3
CTG Pancreatic Cancer—see, for example, FIG. 2
CTG Lung Cancer—see, for example, FIG. 4

### Discussion

Safe—no significant weight loss, no death. See, FIG. 1, for example.
Greatest response in pancreatic cancer intraperitoneal group
Intraperitoneal vs subcutaneous
   Foreign body reaction in subcutaneous group

### REFERENCES CITED IN THIS EXAMPLE

Sofia A. Tsvetikova, Elena I. Koshel, Microbiota and cancer: host cellular mechanisms activated by gut microbial metabolites. International Journal of Medical Microbiology, Volume 310, Issue 4, 2020, 151425, ISSN 1438-4221 doi.org/10.1016/j.ijmm.2020.151425.
Mirzaei R, Afaghi A, Babakhani S, et al. Role of microbiota-derived short-chain fatty acids in cancer development and prevention. Biomedicine & Pharmacotherapy. 2021; 139:111619. doi:10.1016/j.biopha.2021.111619.
Yang Q, Ouyang J, Sun F, Yang J. Short-Chain Fatty Acids: A Soldier Fighting Against Inflammation and Protecting From Tumorigenesis in People With Diabetes. Front Immunol. 2020; 11:590685. Published 2020 Dec. 8. doi:10.3389/fimmu.2020.590685

### EQUIVALENTS

Those skilled in the art will recognize, or be able to ascertain, using no more than routine experimentation, numerous equivalents to the specific substances and procedures described herein. Such equivalents are considered to be within the scope of this invention and are covered by the following claims.

1.-44. (canceled)

45. A method of treating cancer in a subject in need thereof, the method comprising administering to the subject a therapeutically effective amount of a pharmaceutical composition, the pharmaceutical composition comprising a combination of short-chain fatty acids, wherein the combination of short-chain fatty acids comprises acetic acid or a pharmaceutically acceptable salt thereof, propionic acid or a pharmaceutically acceptable salt thereof, and butyric acid or a pharmaceutically acceptable salt thereof.

46. The method of claim 45, wherein the combination of short-chain fatty acids comprises by weight about 60% of acetic acid or a pharmaceutically acceptable salt thereof, about 25% propionic acid or a pharmaceutically acceptable salt thereof, and about 15% butyric acid or a pharmaceutically acceptable salt thereof.

47. The method of claim 46, wherein the combination of short-chain fatty acids comprises by weight about 60% sodium acetate, about 25% sodium propionate, and about 15% sodium butyrate.

48. The method of claim 45, wherein the pharmaceutical composition further comprises a pharmaceutically acceptable excipient.

49. The method of claim 45, wherein the cancer is colon cancer, pancreatic cancer, or lung cancer.

50. The method of claim 45, wherein the pharmaceutical composition reduces viability of a cancer cell of the subject.

51. The method of claim 45, wherein the pharmaceutical composition reduces viability of a cancer cell of the subject by at least about 2%.

52. The method of claim 45, wherein the pharmaceutical composition induces necrosis of a tumor of the subject.

53. The method of claim 45, wherein the pharmaceutical composition reduces size of a tumor in the subject.

54. The method of claim 45, further comprising administering another therapeutic agent in combination with the pharmaceutical composition.

55. A method of inhibiting viability of a cancer cell in a subject, the method comprising administering to the subject a therapeutically effective amount of a pharmaceutical composition, the pharmaceutical composition comprising a combination of short chain fatty acids, wherein the combination of short chain fatty acids comprises acetic acid or a pharmaceutically acceptable salt thereof, propionic acid or a pharmaceutically acceptable salt thereof, and butyric acid or a pharmaceutically acceptable salt thereof.

56. The method of claim 55, wherein the combination of short-chain fatty acids comprises by weight about 60% of acetic acid or a pharmaceutically acceptable salt thereof, about 25% propionic acid or a pharmaceutically acceptable salt thereof, and about 15% butyric acid or a pharmaceutically acceptable salt thereof.

57. The method of claim 55, wherein the combination of short-chain fatty acids comprises by weight about 60% sodium acetate, about 25% sodium propionate, and about 15% sodium butyrate.

58. The method of claim 55, wherein the pharmaceutical composition further comprises a pharmaceutically acceptable excipient.

59. The method of claim 55, wherein the cancer is colon cancer, pancreatic cancer, or lung cancer.

60. The method of claim 55, wherein the pharmaceutical composition induces necrosis of a tumor of the subject.

61. A composition comprising a combination of short chain fatty acids, wherein the combination of short-chain fatty acids comprises by weight about 60% of acetic acid or a pharmaceutically acceptable salt thereof, about 25% propionic acid or a pharmaceutically acceptable salt thereof, and about 15% butyric acid or a pharmaceutically acceptable salt thereof.

62. The composition of claim 61, wherein the combination of short-chain fatty acids comprises by weight about 60% sodium acetate, about 25% sodium propionate, and about 15% sodium butyrate.
